(19) United States
(12) Patent Application Publication
(10) Pub. No.: US 2004/0038914 A1
(43) Pub. Date: Feb. 26, 2004

(54) Flavone/ flavanone derivatives for the systemic treatment and prophlaxis of uv-induced dermatoses

(76) Inventors: Jean Krutmann, Wegberg (DE); Helger Stege, Freiburg (DE)

Correspondence Address: MERCHANT & GOULD PC, P.O. BOX 2903, MINNEAPOLIS, MN, 55402-0903, US

(21) Appl. No.: 10/312,105

(22) PCT Filed: Jun. 28, 2001
(86) PCT No.: PCT/DE01/02285

(30) Foreign Application Priority Data
Jun. 28, 2000 (DE) 10031457.0

(51) Int. Cl. A61K 31/7048; A61K 31/353; A61K 7/42

(52) U.S. Cl. 514/27; 514/456; 424/59

(57) ABSTRACT

The invention relates to the use of flavone/flavanone derivatives of general fortnula (I) for the production of medicaments for the systematic treatment and prophylaxis of UV-induced dermatoses, in particular polymorphic light dermatoses and the sub-types thereof and/or the undesired long-term consequences of UV-radiation, in particular light-aging. The invention further relates to the use of the compounds of general formula (I) for the systematic treatment and prophylaxis of the above UV-induced dermatoses.

### DESCRIPTION

The invention concerns the use of flavone/flavanone derivatives of the general formula I

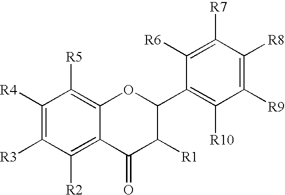

in which independent of one another

R1H, OH or —O-Gly;

R2, R6, R9 and R10 can be the same or different and H, OH, alkoxy, hydroxyalkoxy or C₃-C₇-cycloalkoxy;

R3 H, C₁-C₄ alkyl or C₃-C₇-cycloalkoxy;

R4 H, OH, —O-Gly, alkoxy, hydroxyalkoxy or C₃-C₇-cycloalkoxy;

R5 H C₁-C₄-alkyl, OH, alkoxy, hydroxyalkoxy or C₃-C₇-cycloalkoxy;

R7 and R8 can be the same or different and H, OH, alkoxy, hydroxyalkoxy, C₃-C₇-cycloalkoxy, (thio(C₁-C₄-)alkyl or —NR11R12; and

R11 and R12 can be the same or different and can mean H or C₁-C₄-alkyl; and wherein

Alkoxy and hydroxyalkoxy can contain a straight chain or branched alkyl group with 1-18 C-atoms; and wherein

Gly can be present or absent and designate a mono- or oligoglycidic residue;

in conjunction with the usual auxiliary and additive substances for the production of medicaments for the systeric treatment and prophylaxis of LV-induced dermatoses, in particular polymorphic light dermatosis, and its sub-forms and/or undesired long term sequelae of UV-radiations, particularly light aging,

as well as the use of the compounds of the general formula I for treatment, and/or prophylaxis of the mentioned UV-induced dermatoses by oral, rectal or parenteral dispensing.

In the compounds of general formula I, Gly is preferably selected from the group of hexosyl or pentosyl residues, independently of each other. Preferred are rhamnosyl or glucosyl residues. However, other glucosyl residucs can be advantageous, as for example allosyl, altrosyl, galactosyl, gulosyl, idosyl, mannosyl, rutinosyl or talosyl.

UV-induced dermatoses, also called photodermatoses, are non-infectious, inflammatory skin diseases whose causal genesis is for the most part still largely unkown. From the clinical viewpoint, one differentiates polymorphic light dermatosis, which can appear in different intensities, solar urticaria, erythematous lupus, prurigo estivalis and others. The most frequent UV-induced dermatosis is polymorphic light dermatosis (in the following abbreviated PLD). Sufferng from this are 10 to 20% of the Caucasian population in the central European countries. This disease occurs at different rates of incidence in all races. Although precise epidemiological studies are lacking, women seem to be afflicted mnore frequently than are men. PLD is covered with the most varied of synonyms: sun allergy, Majorca acne, etc.

Typically, in the case of this UV-induced dermatosis one finds a different phenomenon picture between individuals. In a patient, however, PLD is manifested strongly monomorphically. Differentiated are the following sub-types: plaque type, erythema-exudative-multiform-like type PLD, vesiculobullous type PLD, henrmorrbagic type PLD, fixed PLD. The PLD light dermatosis shows histologically an inflammatory, partially perivascular infiltrate of lymphocytes. Immunohistochemically, there is an expression of proinflammatory cytokines in the region of the basal membrane and a strengthened expression of proinflammatory cytokins around the vessels. The pathogenesis of PLD is not yet completely clarified, however it does appear to be clear that the pathologically increased reaction to UV-rays is a consequence of a UV-occasioned release of acid radicals. By this means, signal transducing paths in the cell will be subsequently altered. The pathogenically deciding moment is, therefore, the releasing or the generation of oxygen radicals.

The usual therapy of UV-induced dermatoses consists in the topical or systemic use of glucocortico-steroids. Appearing here are steroid side effects, as for example atrophy of the skin, formation of telean-giectases and the danger of a hypertrichosis. Results of therapy after systemic dispensing of antihistamines, vitamins or electrolytes were anecdotally reported. These positive therapeutic effects could not, however, be confirmed in controlled studies.

More meaningful and desired in greater measure by patients are prophylactic measures, i.e. methods that can be used so that UV-induced dermatoses can not be manifested at all.

In the prophyaxis of photodermatoses, employed for one thing are phototherapeutic methods, i.e. the devil is driven out with Beelzebub. This statement is supported by the experience that the triggering of UV-induced dermatoses weakens after repetitive UV exposures, and that most patients suffer from skin changes in early summer or at the beginning of their vacation, while the appearance of skin changes is more seldomly observed in late summer. However, hardening therapy is extremely time consuming, it requires regular, in part multi-month irradiations by a photodermatologically-schooled and experienced expert (dermatologist), and shows a therapeutic efficacy in only about 30% of patients.

Another possibility is the topical application of sunscreen creams. This measure, relative to its efficacy in comparison to phototherapy is actually easier to carry out, also much less effective. In fact, it is such that only selected sunscreen preparations are in a position at all to offer any kind of protection. Here we are dealing with preparations that display an extremely high sunscreen factor. The application of such a high sunscreen factor requires a very great compliance on the part of the afflicted person, since this strong protection function can be achieved only in combination with physical filters. Physical filters are pigments, e.g. titanium or zinc oxides that, even in a microsomal preparation leave a whitish film on the skin. This cosmetic drawback as well as complete impediment to skin pigmentation because of the high light protection factor is for most afflicted persons difficult to accept.

The use of antioxidants in the prophylaxis of PLD is, up until the present, being done only in topical form. One sunscreen cream combination found in the market since a few years contains rutoside derivatives. This sunscreen cream is being developed as a prophylactic preparation for PLD. However, experience shows that the topical application for achieving optimal results requires a pretreatment time of several weeks before a UV-exposure, and meets only slight compliance on the part of the afflicted person. Additionally, the clinical studies carried out a long time ago with this product have yielded that this preparation is only of limited efficacy. Thus, the development of skin changes in the case of PLD patients is not completely impeded, rather merely weakened. Presumably, this is to be ascribed to the fact that the topical application way is not optimal for achieving a sufficient quencher concentration in the skin layers, in which the formation of oxygen radicals is of pathogenic significance.

Basically, for topical use of sunscreen creams in general and topical antioxidants in particular, to be said is that external use has the following disadvantages:

1.) topical application over large body areas is extremely annoying for the afflicted person, and is consequently not often carried out (time consuming, application must be carried out at least 20 minutes before UV exposure, frequently an unpleasant feeling of the cream-covered skin),

2.) the efficacy of topical preparations is reduced by side effects such as e.g. lack of waterproofness (sea water, fresh water, sweat) as well as reduced by abrasion through textiles and movement,

3.) high sunscreen factors through physical filters have slight cosmetic acceptance, since they form a visible film on the skin,

4.) high sunscreen factors prevent effective pigmentation, this is as a rule undesirable,

5.) frequent use of chemical sunscreen factors increases the risk of an epicutaneous sensitization, since many sunscreen factors possess a strong sensitizing power; the arising of photoallergic contact eczemas fromn light protection causes is scientifically well documented.

The object of the invention up to now was, therefore, to make available for the patients an acceptable possibility of therapy and/or prophylaxis for the mentioned dermatoses, preferably through oral or parenteral administration of suitable active substances and/or their pharmaceutical forms of preparation.

Amazingly, it has now been found that flavone/flavanone derivatives of the general formula I in systemic medicaments excellently fulfill this objective. Flavones and/or flavanones display their action by the fact that they can quench (buffer off) UV-induced oxygen radicals. Particularly well suited for this are rutosides or rutoside derivatives. Additionally, flavone, particularly rutosides or rutoside derivatives also act on vessels by displaying there a clearly endothelial plugging action. This property is already utilized by the dispensing of rutosides as a protection against edema. Flavones, particularly rutosides or rutoside derivatives are spoken of as having anti-inflammatory properties.

Undesirable side effects in the case of these preparations are to expected only in slight measure. To be mentioned in particular, however, are increases in transient transaminases. In the case of rutoside-containing medicaments, slight gastrointestinal side effects are described in about 1% of patients.

Preferred flavone/flavanone derivatives of the general formula I are, for example:

1.) Rutin, (Rutoside; quercetin-3-rutinoside, 3-[[6-O-(6-deoxy-α-L-mannopyranosyl)-beta-D-glucopy-ranosyl]oxy]-2-(3,4-dihydroxyphenyl)-5,7-dihydroxy-4H-1-benzopyran-4-on); as well as its aglycone

2.) O-β-hydroxyethylrutoside, a mixture of mono-, di-, tri- and tetrahydroxyethyl derivatives of rutin; or their algycons; as well as the main component of O-β-hydroxyethylrutoside, namely

3.) Troxerutin-(2[3,4-bis(2-hydroxyethoxy)phenyl]-3-[[6-O-(6-deoxy-α-L-mannopyranosyl)-β-D-glucopyranosyl]oxy]-5-hydroxy-7-(2-hydroxyethoxy)-4H-1-benzopyran-4-on); as well as its aglycon;

4.) Monoxerutin-(2-[3,4-bishydroxyphenyl]-3-[[6-O-(6-deoxy-α-L-mannopyranosyl)-β-D-glucopyranosyl]oxy]-5-hydroxy-7-(2-hydroxyethoxy)-4H-1-benzopyran-4-on); as well as its aglycon;

5.) α-glucosylrutin;

6.) Naringin 7-[[2-O-(6-deoxy-α-L-mannopyranosyl)-β-D-glucopyranosyl]oxy]-2,3-dihydro-5-hydroxy-2-(4-hydroxyphenyl)-4H-1-benzopyran-4-on); as well as its aglycon naringenin;

7.) Hesperidin (7-[[6-O-(6-deoxy-α-L-mannopyranosyl)-β-D-glucopyranosyl]oxy]-2,3-dihydro-5-hydroxy-2-(3-hydroxy-4-methoxyphenyl)-4H-1-benzopyran-4-on; as well as its aglycon hesperetin;

8.) Diosmin (3′5,7-trihydroxy-4methoxyflavone-7-rhamnoglucoside; as well as its aglycon diosmetin;

9.) dihydrorobinetin (3,3′,4′,5′,7-pentahydroxyflavanone);

10) Taxifolin (3,3′,4′,5,7-pentahydroxyflavanone);

11) Eriodictin (3′,4′,5,7,-tetrahydroxyflavanone-7-rhamnoside); as well as its aglycon eriodictyol;

12) Flavanomarein (3′,4′,7,8-tetrahydroxyflavanone-7-glucoside); as well as its aglycon;

13) Isoquercetin (3,3′,4′,5,7-pentahydroxyflavanone-3-(β-D-pyranoside); as well as its aglycon;

14) Leucocyanidin (3,3′,4,4′,5,7-hexahydroxyflavan);

15) Cyrtominetin (6-8-dimethyl-3′,4′,5,7-tetrahydroxyflavanone);

16) 6,8-dimethyl-5,7-dihydroxy-4′-thiomethylflavanone;

17) 6,8-dimethyl-4′,5,7-trihydroxy-3′-methoxyflavanone; or

18) 6,8-dimethyl-5,7-(dihydroxy-4′-dimethylamino)-flavanone.

The flavone/flavanone derivatives of the general formula I can be used in usual medicinal preparations in solid or liquid form, as for example powder, soft or hard capsules, tablets, pills, bath tablets, suppositories, emulsions, oils, solutions or lyophilisates, with the usual auxiliary and additive substances.

Solutions or lyophilisates can also be systemically administered parenterally in the form of injections as dissolved ready-to-use preparations or as lyophilsates, to which is added a thinning solution, e.g. isotonic NaCl-solution or injectable water.

The advantages of a systemic enteral or parenteral dosing compared to a topical application are:

1) greater efficacy,

2) distinctly better compliance,

3) uniform tissue level,

4) also greater bioavailability in tissue compartments, which can not be attained by a topical application.

In investigations of afflicted persons, shown in surprising fashion was that medicatnents that contain rutoside or ruteside esters or a high proportion of other flavones, with exception of nicotine amide, are capable of completely preventing outbreak of a PLD and that this property could also be observed in the case of those afflicted persons for whom, up to now, all other known methods have failed. The following casuistics make clear that the efficacy of these medicaments is superimposed on the efficacy of topical or physical therapies.

### CASE EXAMPLES

### Case 1

Female patient, 50 years, PLD, PLD known for 20 years.

Previous therapies:

1. Conventional sunscreen agents; without effect.

2. Hardenig therapy with PUVA (Psoralens long wave ultraviolet radiation) worsening of symptoms, therefore continuation not possible.

Because of the severity of the progression, presentation of this female patient during our special discussion hour for photodernatoses.

Results of photo tests in our clinic:

Initial testing: November 1998:

Immediate pigment darkening (=UVA [long wave ultraviolet radiation] immediate pigmentation): 60 J/cm².

Minimum erythema dosing (=UVB [shortwave ultraviolet radiation] sensitivity: 100 mJ/cm².

Photo provocation testing

Upper back UVA:

It was possible to carry out only a 2-time radiation (usually 3 times) since there was very severe pain in the test area, and on day 3 there was development of PLD with papules, eiythema and itching.

Upper back, UVA+UVB:

It was likewise possible to perform only a 2-time radiation, after which immediate burning and itching.

Diagnosis: UVA-triggered PLD

Retesting May 5, 1999:

Photo provocation testing (buttocks):

Massive erythema reaction wit infiltrate, buring and itching after the first radiation, thereupon broke off testing.

Jun. 5, 1999: Introduction of therapy with a rutoside-containing preparation (daily dosing; 3000 mg O-β-hydroxyethylrutoside; 3×2 tbl/day for a total of 2 weeks)

Renewed photo provocation on May 25, 1999:

3-time radiation with UVA was tolerated without problems this time. Erythema appeared only after the third radiation, without papules or infiltration.

After this, continuation of the therapy until today. The patient states being able to stand the sun again and, except for normal sunscreen agents, does not have to resort to any measures for protection against the sun.

Comments: This case verifies the efficacy of an orally administered rutoside for PLD prophylaxis. It is important that a clearly greater action could be achieved through this preparation than through the use of topical sunscreen agents or phototherapy.

### Case 2

Male patient, 42 years old,

Since 1980, anamnestically-triggered UVA, since skin changes also appear after sun exposure through window glass (e.g. while driving).

Previous therapies:

Sun screen agents: at first effective, now without effect for two years, topical rutoside-containing preparation. No improvement.

Hardening therapy (PUVA): ineffective.

Patient has been treated in our clinic since 1980, comes in regularly with the question concerning new therapeutic options, hence renewed testing.

Photo provocation of 2^{nd}, 3^{rd} and 4^{th} of June 1999.

Induction of PLD on upper back with UVA, UVB and UVA+UVB (development of infiltrated erythema and papules (primarily in the case of UVA+UVB); diagnosis PLD.

Therapy with a rutoside-containing medicament (daily dosing: 3000 mg O-β-hydroxyethylrutoside, 3×2 tbl./day for a total of 2 weeks.

Renewed photo provocation on Jun. 28-30, 1999:

Again upper third of the back directly below the preceding test area; now no induction of papules, weakened erythema reaction, no indication toward PLD.

Comments: This case emphatically verifies that through systemic administration of a rutoside it was possible the first time in the case of this patient effectively to prevent a PLD. Conventional measures (topical sunscreen including topical rotuside, hardening therapy) were ineffectual.

### Case 3

Female patient with a bullous form of PLD known for years, that additionally displayed urticarial characteristics. Preliminary investigations yielded a reproducible triggering of skin changes under photodermatological test conditions with 3 repetitive UVA-1 radiations (340-400 nm). After intravenous administration of a rutoside-ester-containing preparation that is approved as (under the name of) Venentoniktrm as a medicament, shown was a complete suppression of the skin changes previously capable of being triggered by UV radiation. This prophylactic property could also be demonstrated under environmental conditions within the scope of sun exposure out in the open over several days.

This case emphasizes in particular the essentially greater efficacy of a systemic rutoside treatment in the case of prophylaxis of PLD, in comparison to topical methods.

1. Use of flavone/flavanone derivatives of the general formula I 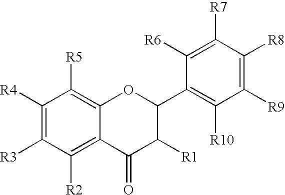
in which independent of one another
R1 H, OH or —O-Gly;
R2, R6, R9 and R10 can be the same or different and H, OH, alkoxy, hydroxyalkoxy or C₃-C₇-cycloalkoxy;
R3 H, C₁-C₄ alkyl or C₃-C₇-cycloalkoxy;
R4 H, OH, O-Gly, alkoxy, hydroxyalkoxy or C₃-C₇-cycloalkoxy;
R5 H C₁-C₄-alkyl, OH, alkoxy, hydroxyalkoxy or C₃-C₇-cycloalkoxy;
R7 and R8 can be the same or different and H, OH, alkoxy, hydroxyalkoxy, C₃-C₇-cycloalkoxy, thio(C₁-C₄-)alkyl or —NR11R12; and
R11 and R12 can be the same or different and can mean H or C₁-C₄-alkyl; and wherein
Alkoxy and hydroxyalkoxy can contain a straight chain or branched alkyl group with 1-18 C-atoms; and wherein
Gly designates a mono- or oligoglycidic residue;
in conjunction with the usual auxiliary and additive substances for the production of medicaments for the systemic treatment and prophylaxis of UV-induced dermatoses, in particular polymorphic light dermatosis, and their sub-forms and/or undesired long term sequelae of UV-radiations, particularly light aging,

2. Use in accordance with claim 1 of rutin or its aglycon, O-β-hydroxyethylrutoside or its aglycon, troxerutin or its aglycon, monoxerutin or its aglycon, α-glycosylrutin, naringin, naringenin, hesperidin, hesperetin, diosmin, diosmetin, dihydrorobinctin, taxifolin,, eriodictin, eriodictyol, flavanomarein or its aglycon, isoquercetin or its aglycon, leucocyanidin, cyrtominern, 6,8-dimethyl-5-7-dihydroxy-4′-thiomethylflavanone, 6,8-dimethyl-4′,5,7-trihydroxy-3′-methoxyflavanone and/or 6,8-dimethyl-5,7-dihydroxy-4′-(dimethylarnine)-flavonone, as well as the aglycons of glycolized compounds.

3. Use in accordance with claims 1 or 2 for producing medicaments containing per daily dose 120 to 3000 mg of flavone/flavanone derivatives in accordance with claim 1 or 2.

4. Use in accordance with claims 1 to 3, characterized in that the active substance of formula I is rutin.

5. Use in accordance with claims 1 to 3, characterzed in that the active substance of formula I is O-β-hydroxyethylrutoside.

6. Use of compounds of the general formula I in accordance with Claim 1 or 2 for the systemic treatment and prophylaxis of UV-induced dermatoses, in particular of polymorphic light dermatosis and its sub-forms and/or undesired long term sequalae of UV radiations, particularly light aging.

7. Use of rutin in accordance with claim 6.

8. Use of O-β-hydroxyethylrutoside in accordance with claim 6.

9. Treatment and prophylaxis of UV-induced dermatoses, in particular polymorphic light dermatosis and its sub-forms and/or undesired long-term sequelac of UV radiations, in particular light aging though oral, parenteral or rectal dispensing of medicament-containing compounds of the general formula I.

10. Treatment and prophylaxis of UV-induced dermatoses in accordance with claim 9 through a daily dosing of 120 to 3000 mg of the compounds of the general formula I.
